(19) United States
(12) Patent Application Publication
(10) Pub. No.: US 2025/0006335 A1
(43) Pub. Date: Jan. 2, 2025

(54) PATIENT TREATMENT RECOMMENDATIONS

(71) Applicant: International Business Machines Corporation, Armonk, NY (US)

(72) Inventors: Tigran Tigran Tchrakian, Castleknock (IE); Sergiy Zhuk, Dublin (IE); Djallel BOUNEFFOUF, Poughkeepsie, NY (US); Mykhaylo Zayats, Dublin (IE); JEFFREY L. ROGERS, Briarcliff Manor, NY (US)

(21) Appl. No.: 18/215,960

(22) Filed: Jun. 29, 2023

(51) Int. Cl. G16H 20/17; G16H 10/60

(52) U.S. Cl. CPC G16H 20/17; G16H 10/60

(57) ABSTRACT

Data associated with a patient undergoing medical treatment can be received. The data can pertain to a use of the medical treatment and a state of the patient. A subset of the data pertaining to the state of the patient can be identified as context. A function can be learned that relates the context to a reward derived from the medical treatment. The function can be used, based on a current state of the patient, to identify a type of treatment to deliver to the patient.

### BACKGROUND

The present application relates generally to computers and computer applications, and more particularly to design and delivery via one or more communication networks, of individualized treatment recommendations of treatments, and/or of recommendations of treatments which can be administered via wearable medical devices.

### BRIEF SUMMARY

The summary of the disclosure is given to aid understanding of a computer system and method of treatment recommendations, and not with an intent to limit the disclosure or the invention. It should be understood that various aspects and features of the disclosure may advantageously be used separately in some instances, or in combination with other aspects and features of the disclosure in other instances. Accordingly, variations and modifications may be made to the computer system and/or their method of operation to achieve different effects.

A computer-implemented method, in some embodiments, includes receiving data associated with a patient undergoing a medical treatment, the data pertaining to use of the medical treatment and a state of the patient. The method also includes identifying a subset of the data pertaining to the state of the patient as context. The method further includes learning a function that relates the context to a reward derived from the medical treatment. The method also includes using the function based on a current state of the patient to identify a type of treatment to deliver to the patient.

A system, in some embodiments, includes at least one processor. The system also includes at least one memory device coupled with at least one processor. At least one processor is configured to receive data associated with a patient undergoing a medical treatment, the data pertaining to use of the medical treatment and a state of the patient. At least one processor is also configured to identify a subset of the data pertaining to the state of the patient as context. At least one processor is also configured to learn a function that relates the context to a reward derived from the medical treatment. At least one processor is also configured to use the function based on a current state of the patient to identify a type of treatment to deliver to the patient.

A computer readable storage medium storing a program of instructions executable by a machine to perform one or more methods described herein is also provided.

Further features as well as the structure and operation of various embodiments are described in detail below with reference to the accompanying drawings. In the drawings, like reference numbers indicate identical or functionally similar elements.

### BRIEF DESCRIPTION OF THE DRAWINGS

FIG. 1 shows an example of a computing environment, which can implement treatment recommendations in some embodiments.

FIG. 2 is a flow diagram illustrating a method in some embodiments that provides treatment recommendations.

FIG. 3 is a diagram illustrating components of a system in some embodiments that provides treatment recommendations.

FIG. 4 is another flow diagram illustrating a method of providing treatment recommendations in some embodiments.

FIG. 5 is a diagram illustrating computer components which can implement provision of treatment recommendations in some embodiments.

### DETAILED DESCRIPTION

A computer-implemented method, in some embodiments, includes receiving data associated with a patient undergoing a medical treatment, the data pertaining to use of the medical treatment and a state of the patient. The method also includes identifying a subset of the data pertaining to the state of the patient as context. The method further includes learning a function that relates the context to a reward derived from the medical treatment. The method also includes using the function based on a current state of the patient to identify a type of treatment to deliver to the patient.

Identifying a type of treatment to deliver to the patient based on the current state can customize the treatment for a patient in particular state, and can provide for a more effective delivery of the medical treatment.

One of more of the following features are separable or optional from each other. In an aspect, the medical treatment is a self-administered medical treatment where the medical treatment is initiated in clinic and subsequently self-applied by the patient. In this way, for example, effective medical treatment can be automatically determined and/or delivered even while the patient is away from the clinic.

In an aspect, the medical treatment is performed using a wearable medical device that the patient is wearing. In this way, for example, changes to the patient's states can be automatically received via one or more sensors of the wearable medical device.

In another aspect, the reward is related to a metric structured as a multi-dimensional vector that describes the state of the patient related to the medical treatment the patient is undergoing. In this way, for example, by having a machine interpretable format, the benefit of the treatment can be assessed as related to the state of the patient by a machine.

In yet another aspect, the reward is a scalar function of the context. In this way, the reward that is related to the context can be determined.

In another aspect, the subset of data includes a set of features from the received data. The set of features can be used in machine learning, for example, reinforcement learning to learn to make a medical treatment recommendation.

In still another aspect, the function is learned using a contextual multi-armed bandit robust with respect to super-gaussian noise. In this way, decision making process can be learned taking into account the state of the patient, e.g., context.

A system including at least one computer processor and at least one memory device coupled with the at least one computer processor is provided, where the at least one computer processor is configured to perform one or more methods described herein. A computer program product that includes a computer readable storage medium having program instructions embodied therewith, the program instructions readable by a device to cause the device to perform one or more methods described herein is also provided.

Medical treatments may involve initial administration by a physician or clinician, followed by self-administration by the patient. For example, a patient can be implanted with or given a treatment device, which can be programmed to provide treatments to the patent. Thereafter, the patient may self-administer the treatment, for example, at home or at another location away from the physician or clinician. Appropriate type or instance of such a treatment at any given time may depend on several variables describing the current state of the patient. In some embodiments, the relationship between this state and the most appropriate instance of the treatment is learned and recommendations are provided.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as medical treatment recommendation algorithm code 200. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

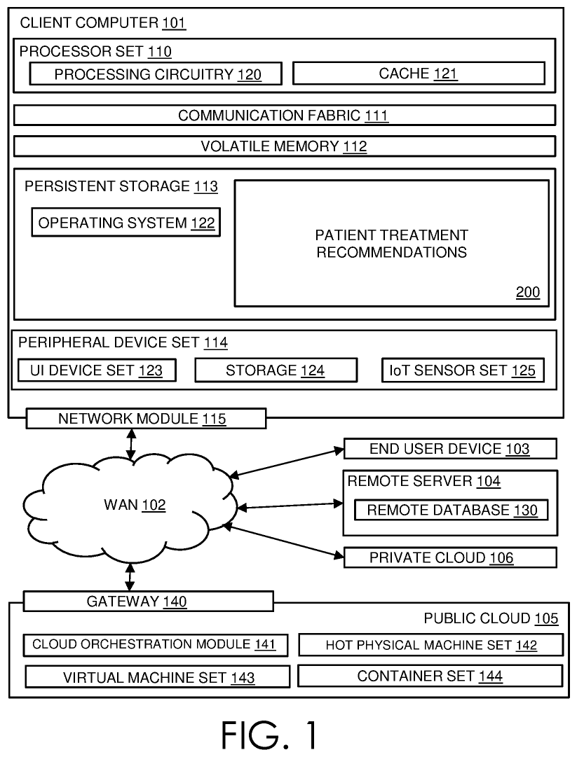

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up buses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way. EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

Systems, methods, and techniques can be provided that can design and deliver individualized treatment recommendations, e.g., medical treatments, for patients. Throughout the description, it should be understood that, with reference to any collection of information or data from patients or others, such collection is performed with the permission of the patients or other users. Thus, the patients or and/or others participate in an opt-in and/or opt-out basis or manner.

Certain types of treatments are initiated in clinics and then subsequently, are applied by the patients, for example, at the patients' homes or other locations outside of the clinics where the initial treatment took place. For instance, a patient may get an implant of a medical device or another wearable device at a clinic, which the patient can subsequently control with a control device such as a remote control device at another location. With the implanted device or another wearable device, the patient may also receive a set of programs that the patient can use via the remote control device that allows the patient to change the stimulus character or treatment character that the patient is receiving through the implanted or wearable device. Via remote data transmission from the medical device, responses to questionnaires from the patient, another smart device the patient may be wearing (e.g., a smartwatch), systems, methods and/or techniques, in one or more embodiments, can observe and/or record information as to what treatment characters the patient (e.g., which one of the program settings) is applying with the medical device, and also how the patient is reacting to those treatments over time, for example, how the patient's condition is evolving as the patient self-administers the treatment. For example, one or more patient's devices can transmit data including such information to a processor of a system disclosed herein, and/or upload the data to a cloud environment. Based on the received data, the processor can learn a relationship between the parameters of the treatment (e.g., how the patient applied the treatment) and the patient's state responding to the application of the treatment over time. Based on the learned relationship, the processor of the system can create a recommendation that can be appropriate for the current state of the patient, and communicate the recommendation to the patient, e.g., via one or more computer and/or telecommunication networks.

In an aspect, an instance of a treatment (e.g., parameters or characters used in the treatment) that is appropriate at a given time for the patient may depend on several variables describing the current state of the patient. The system, for example, learns the relationship between this state of the patient and the most appropriate instance of the treatment. The system then uses the learned relationship to recommend and/or provide, for example, in real time, or substantially in real-time, treatments to the patient which are determined to be appropriate for that given time.

A system, in some embodiments, includes one or more hardware or computer processors that function or operate to provide delivery of individualized treatment recommendations. A processor collects dynamic data pertaining to a patient, e.g., from wearable sensor that can measure physical or bodily conditions, from responses to questionnaires, environmental data, in addition to data describing self-administered treatment, which the patient is undergoing. A processor identifies a subset of the collected patient data (e.g., context) that parameterizes the self-administered treatment. A processor also learns a metric (an individualized metric of a patient) that quantifies the benefit a patient obtains from the treatment (e.g., reward). A processor can learn functions relating the context to the benefit obtained using instances of the treatment that the patient used while within that context. A processor uses the function to recommend specific instances of the treatment to the patient based on the patient's current context.

Context refers to a subset of the patient data or data associated with the patient, including the patient's state that contextualizes the patient and informs the condition of the patient. This subset of the patient data can include environmental data such as atmospheric data and need not be limited to only the patient's physical condition. Individualized metric can indicate, or be referred to as, a reward, e.g., an improvement in quality of life. For instance, every time a treatment is applied, the patient may obtain or gain a reward. That reward can be tied to a specific type of treatment (e.g., an instance of a treatment or program of treatment). A reward can be parameterized by a set of data (context). Individualized metric can be structured as a vector of different parameters that describe the patient such as, on a given day, the patient's pain, mood, number of steps taken, alertness, and other conditions that can describe the patient's overall physiological and psychological state. Reward can be defined in terms of the values of the individualized metric. In an embodiment, a reference state or idealized state values of the metrics can be established and the vector representing the individualized metric can store values representing distances of the parameters from the reference state values. The system learns functions relating the context to the reward, or relating the reward to the context, learn how the reward is parameterized by the context. This can be done for each type of treatment (e.g., each instance, each program option, or characteristic). The learned parameters can be a linear combination of the components of the context, in an embodiment. In another embodiment, the learned parameters can be a non-linear combination of the components of the context. Learning those parameters allows the system to make a prediction and recommendation about each treatment option, for example, each of different programs or instances of a treatment. In an embodiment, the system can choose an instance of a treatment based on the context the patient happens to be under in a given time or day. For example, when providing a treatment recommendation, the context determines the recommendation for a given time or day. For each treatment (instance or program of a treatment), different parameters (e.g., context) can parameterize a patient's individual metric or reward. For example, an instance of a treatment is associated with context that parameterizes an individual metric or reward of a patient. Context that parameterizes an individual metric of a patient can be different for different instances of a treatment. Context indicates or refers to a set of parameters that show the patient's state and is relevant to the recommendation that is being made.

For a given treatment type or option, context or subset of patient data can be identified that have values within a threshold of reference data. For example, a vector of parameters (associated with a patient) can be compared with a reference vector of parameters having reference values, and those parameters that are within the threshold of the reference values can be selected or identified as context (subset of patient data). Reward is assumed to be a function of the context. Hence, reward is parameterized by that subset of patient data.

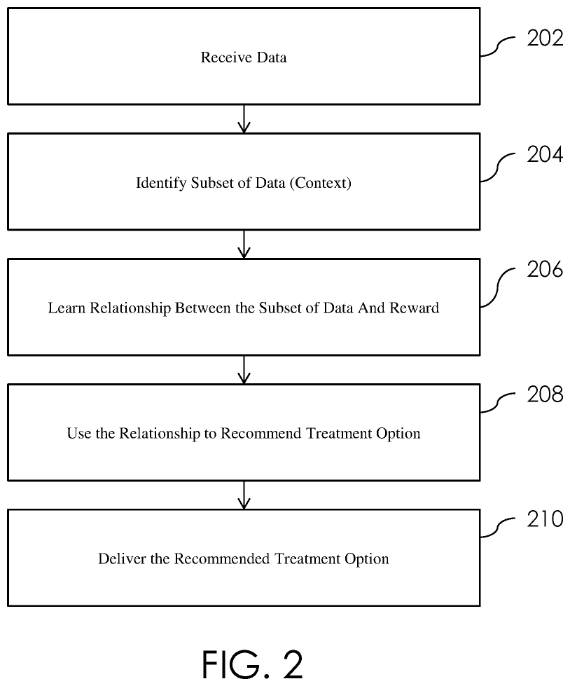

FIG. 2 is a diagram illustrating a method in some embodiments. The method can be performed by and/or implemented on one or more computer processors, which for example, can be one or more processors described above with reference to FIG. 1. At 202, data is received. The data can pertain to, or associated with, a use of a treatment, for example, types or characteristics of the treatment, which a patient is administering on the patient (e.g., self-administering a treatment). There can be multiple treatment types or options. For instance, there can be different programmed treatment options on the device, and the data specifies which programmed treatment option the patient is using to administer the treatment. The data also includes data that characterizes the state of the patient. In some embodiments, the data is a time series data having a time component. For example, in this processing, the method can include receiving data associated with a patient undergoing a medical treatment, e.g., self-administered medical treatment, where the data pertains to a use of the medical treatment and a state of the patient.

At 204, a subset of the patient state data is identified as being relevant to the treatment. For instance, patient state data that is within a threshold of a reference value can be considered relevant to a treatment. This subset of the patient state data is referred to as context. For example, in this processing, the method includes identifying a subset of the data pertaining to the state of the patient as context. By way of example, the context can be determined by running a multiple regression with the reward as the dependent variable and the potential context variables as the independent variables. Doing so can give an indication of how each of the potential context variables affects the reward, allowing for a subset of those variables to be chosen. A context variable that is “relevant” to the treatment refers to one whose value has an impact on the reward of the treatment. For example, if the reward of a treatment is a decrease in pain, the pain before treatment is considered a suitable context variable.

At 206, a function is learned, relating that subset of the patient state data to a reward or benefit derived for specific programmed treatment options or specific instances of the treatment. The function informs how the context (a set of patient state data) is related to the reward or benefit derived from a treatment instance. The function can be expressed in terms of context that parameterizes the reward. A reward to a patient is also referred to as a patient's individualized metric. Thus, for example, context is identified, which parameterizes an individualized metric. For example, in this processing, the method includes learning a function that relates the context to a reward derived from the medical treatment, e.g., the self-administered medical treatment. By way of example, the function can be linear (e.g., a regression function) and the parameters can be learned during the filtering.

At 208, using the learned function or relationship and based on the patient's current state, e.g., current context, treatment recommendations can be identified to deliver to the patient. By way of example, a Contextual Multi-armed Bandit (CMAB) can be trained. Having trained the recommender, recommendations can be generated based on the current context. For instance, if a treatment option or a specific type of treatment with beneficial result (reward) was given to the patient at the time when the patient's state was similar to that of the current context, then that treatment option or that specific type of treatment can be recommended. For instance, a treatment option or a type of treatment with the highest degree of reward that is associated with the patient's current context, can be selected for recommendation. For instance, in this processing, the method includes using the function based on a current state of the patient to identify a type of treatment to deliver to the patient. By way of example, treatment can be an adjustment to a medication dosage level.

At 210, the method also includes delivering the recommended treatment option. For instance, a recommended treatment option can be transmitted via a communication network to a patient's device such as smartphone, laptop, e.g. via email or text. In another aspect, the recommended treatment option can be transmitted to the patient's physician or another care giver. Yet in another aspect, the recommended treatment option can be transmitted to the patient's wearable medical device, where the option is available for administering by the patient, for example, once approved or verified by the physician. In another aspect, once approved, the patient's wearable medical device can be automatically caused to administer the recommended treatment option.

In some embodiments, the medical treatment is a self-administered medical treatment where the medical treatment is initiated in clinic and subsequently self-applied by the patient. In some embodiments, the medical treatment is performed using a wearable medical device that the patient is wearing. In some embodiments, the reward is related to a metric structured as a multi-dimensional vector that describes the state of the patient related to the medical treatment the patient is undergoing. In some embodiments, the reward is a scalar function of two of more elements of the metric. In some embodiments, the subset of data includes a set of features from the received data. In some embodiments, the function is learned using a contextual multi-armed bandit robust with respect to super-gaussian noise.

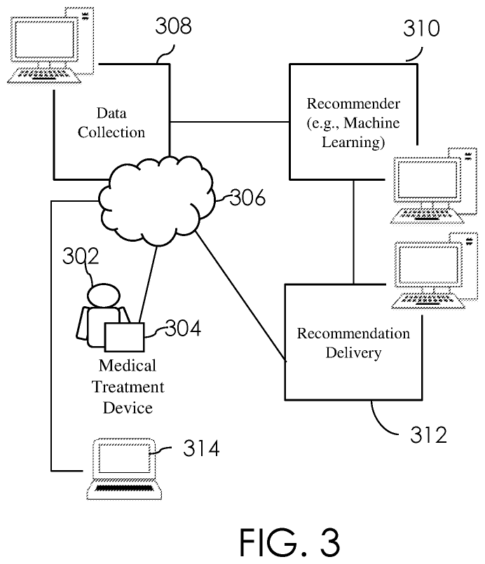

FIG. 3 is a diagram illustrating components of a system in an embodiment. The components can be modular or functional components of a computer system, and can for example be logical components such as software functions. Those components can be implemented on one computer processor or a plurality of computer processors. A patient 302 undergoes a self-administered treatment using a medical device 304, e.g., which the patient is wearing. Data collection component 308 collects data related to the treatment (e.g., data pertaining to the use of the treatment), and the patient state data (e.g., data characterizing the state of the patient), for example, continually over time while the patient is undergoing treatment. Thus, in some embodiments, the data is a time series data. For instance, the medical treatment device 304 continually and/or automatically uploads the data to a data collection computer, e.g., a cloud environment where the data can be stored, e.g., via remote communication networks such as the Internet. The patient responds to questionnaires and posts the responses to the data collection computer or component 308, for example, using a patient's computer device 314 or smartphone device or another device, via the communication networks 306. For example, data collection can be done through a combination of sensors, questionnaires and environmental data, as well as other any data collection mechanism related to the treatment that the patient is undergoing.

A recommender component 310 identifies a subset of continually collected patient data (context) that parameterizes a reward obtained from a self-administered treatment that the patient is undergoing. The recommender component 310 also learns a function relating this context to the benefit obtained using an instance of the above treatment that the patient used while within that context. For example, a subset of the patient state data is identified as being relevant to the treatment, and a function is learned, relating that subset of the patient state data (parameters) to the benefit derived for specific instances of the treatment. Using the function, a recommended treatment is identified. In an embodiment, reinforcement learning can be employed in learning the function. For example, contextual multi-armed bandit with new noise assumption (zero mean with bounded second moments) is employed.

A recommendation delivery component 312 delivers treatment recommendations to patients given their current context or based on their current state. For example, given the current context of patient 304, the recommendation delivery component 312 uses the learned function to determine a recommended treatment to the patient and delivers the treatment. Delivery of the treatment can occur in different forms, e.g., email or text or another notification to the patient 302 via a patient's device 314, and/or a physician, notification to the medical device 304, and/or others.

Available data collection applications can be utilized by the data collection component 308 to collect data, and available cloud services can be employed for computations associated with the training of the recommender system subsequent to the generation of recommendations.

In multi-armed bandit (MAB) machine learning framework, an agent selects actions (arms) in order to maximize its cumulative reward in the long term. The contextual multi-armed bandit (CMAB) makes a decision conditional on the state of the environment, e.g., the context. In some embodiments, the recommender component 310 employ a contextual multi-armed bandit (CMAB) whose measurement noise assumption is taken to be sub-exponential. This version of CMAB has a new regret bound with non-sub-gaussian noise, that depends on the choice of an information gain function, and a quantity referred to herein as regret-information ratio.

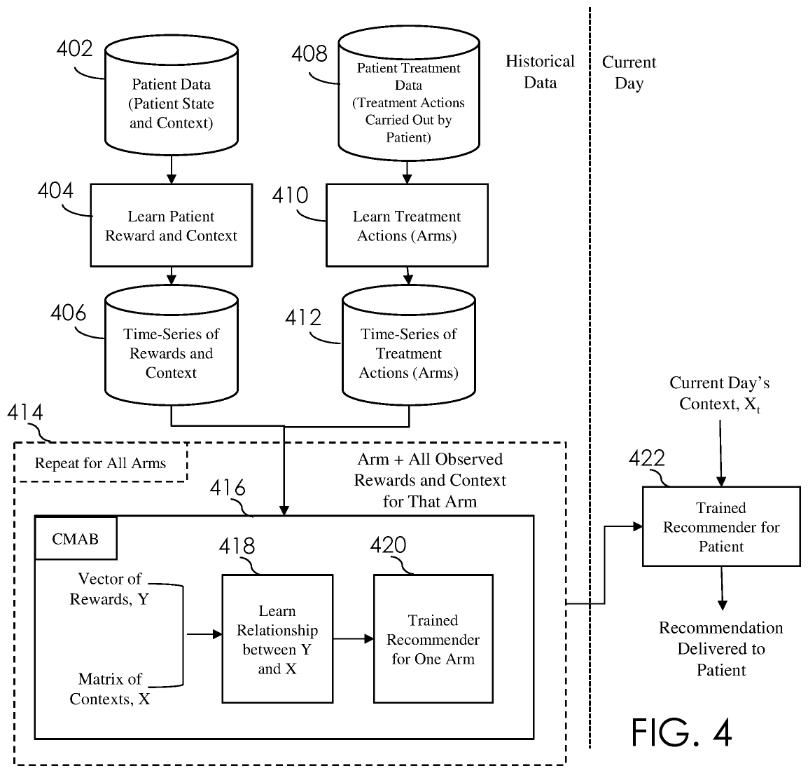

FIG. 4 is another flow diagram illustrating a method of providing treatment recommendations in some embodiments. The method can be implemented on or run on one or more computer processors, e.g., running in a computing environment described above with reference to FIG. 1. At 402, patient data, e.g., patient state and context, is received, accessed, or retrieved, e.g., from a storage device. At 404, using the patient data, patient reward and context are learned. At 406, time-series data of rewards and context is created and stored on a storage device. For example, a database of such time-series data can be created. Similarly, at 408, patient treatment data, e.g., treatment actions carried out by the patient is received, accessed, or retrieved, e.g., from a storage device. At 410, using the treatment data, treatment actions (also referred to as arms) are learned. At 412, time-series data of treatment actions is created and stored on a storage device. For example, a database of such time-series data can be created.

In some embodiments, the data received at 402 and 408 are historical data from which a model can be trained or built. At 414, a contextual multi-armed bandit (CMAB) model is trained, for example, for all arms (treatment actions). For instance, the time-series of treatment actions (arms) and the time-series of rewards and context are used for training the CMAB model 416 for all arms. For instance, a vector of rewards, Y and a matrix of contexts, X is provided for the CMAB to learn a relationship between Y and X as shown at 418 and produce a trained recommender for one arm (a treatment action) 420.

A trained recommender for a given patient trained for all arms (e.g., trained as shown at 414) can be used for providing treatment recommendations on a given day. For instance, current day's context, Xₜ can be fed into a trained recommender for patient 422. Based on the current day's context, Xₜ, the trained recommender for patient 422 predicts a treatment recommendation or recommendations, e.g., that provide the highest reward for that context.

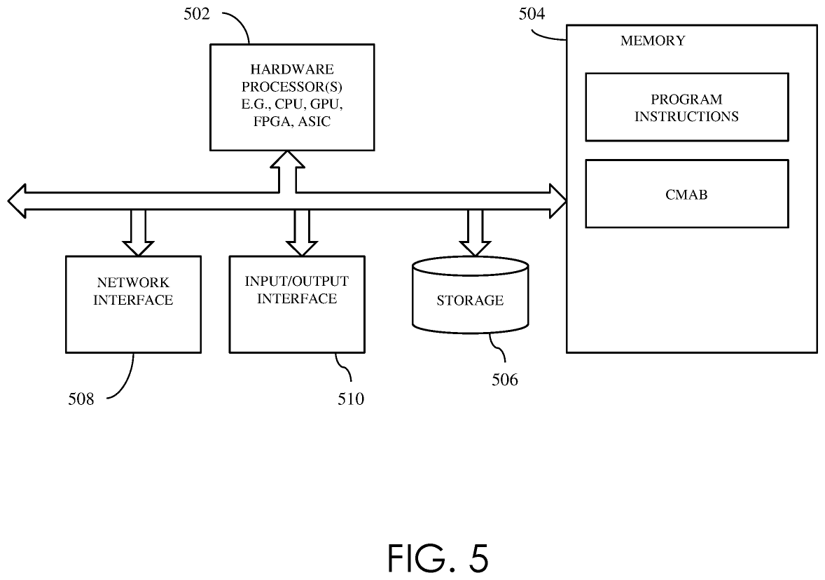

FIG. 5 is a diagram showing components of a system in some embodiments that can provide patient treatment recommendations. One or more hardware processors 502 such as a central processing unit (CPU), a graphic process unit (GPU), and/or a Field Programmable Gate Array (FPGA), an application specific integrated circuit (ASIC), and/or another processor, may be coupled with a memory device 504, and generate a model and recommend treatment options. A memory device 504 may include random access memory (RAM), read-only memory (ROM) or another memory device, and may store data and/or processor instructions for implementing various functionalities associated with the methods and/or systems described herein. One or more processors 502 may execute computer instructions stored in memory 504 or received from another computer device or medium. A memory device 504 may, for example, store instructions and/or data for functioning of one or more hardware processors 502, and may include an operating system and other program of instructions and/or data. One or more hardware processors 502 may receive data associated with a patient undergoing a medical treatment, the data pertaining to use of the medical treatment and state of the patient. One or more hardware processors 502 may identify a subset of the data pertaining to the state of the patient as context. One or more hardware processors 502 may learn a function that relates the context to a reward derived from the medical treatment. One or more hardware processors 502 may use the function based on a current state of the patient to identify a type of treatment to deliver to the patient. Data may be stored in a storage device 506 or received via a network interface 508 from a remote device, and may be temporarily loaded into a memory device 504 for building or generating the recommendation model. The learned model may be stored on a memory device 504, for example, for running by one or more hardware processors 502. One or more hardware processors 502 may be coupled with interface devices such as a network interface 508 for communicating with remote systems, for example, via a network, and an input/output interface 510 for communicating with input and/or output devices such as a keyboard, mouse, display, and/or others.

The system and/or method described herein can be used to provide individualized treatment recommendations, for example, online in real time. There can be less habituation to the treatment for patients that receive the recommended treatments, e.g., less of a diminished response to repeated treatments. Further, treatments can be catered specifically to an individual, and based on a specific reward or benefit for that individual, which can be different among patients.

In some embodiments, a system and/or method provide design and delivery of individualized treatment recommendations for patients. For example, data is collected from patients undergoing medical treatment. A metric is learned for the patients from the data collected. For example, each patient can have a metric, based on the data collected about the patient. By way of example, the metric is learned by taking variables that are expected to be impacted by the treatment, perform clustering in the space spanned by those variables, identifying a “best” cluster, and defining a metric based on its centroid. Features (e.g., context or a subset of the metric) are identified that parameterize a reward based on the metric. A recommender component is trained to provide treatment options that optimize the metric. In some embodiments, the inputs to the training (e.g., a Contextual Multi-armed Bandit (CMAB) model) include the observed rewards and contexts, and the outputs are the parameters that parametrize the function that relates rewards to context.

In some embodiments, the treatment is any medical treatment that has been initiated in a clinic setting and is self-applied by the patient thereafter. In some embodiments, the metric is a multi-dimensional vector that describes aspects of the patient's state relevant to the treatment the patient is undergoing. In some embodiments, the reward is a scalar function of the context. In some embodiments, the features or context are a set of features from the collected data. In some embodiments, the recommender component employs a contextual multi-armed bandit robust with respect to super-gaussian noise.

As described above, a method and/or system can apply to medical treatments that are initiated in-clinic by a physician or clinician and are thereafter self-administered by the patient. Patient data can be input, which can include continually collected data pertaining to the patient's state, and data describing which treatments the has self-administered. For example, an instance of a treatment can have a set of parameter values or attributes, which can be applied via a medical device. These parameter values or attributes can be different for different instances of treatment. Output can include a metric learned from the data (e.g., reward) that describes the success of instances of the treatment the patient is undergoing, a subset of patient data (e.g., context) that parameterizes the “reward” of the treatment that the patient is undergoing, and recommended instances of treatment based on the current “context” of the patient. This recommendation is that which maximizes the “reward.”

In some embodiments, determining recommended treatment uses a reinforcement learning process, which allows the recommendation of the treatment in non-stationary environment with non-stationary information. In some embodiments, recommendation is done online with online interaction with the use of the treatment. An example of a medical recommendation can be pain reliever dosage, where the recommendation adjusts the dosage based on dynamic information collected from the patient.

The terminology used herein is for the purpose of describing particular embodiments only and is not intended to be limiting of the invention. As used herein, the singular forms “a”, “an” and “the” are intended to include the plural forms as well, unless the context clearly indicates otherwise. As used herein, the term “or” is an inclusive operator and can mean “and/or”, unless the context explicitly or clearly indicates otherwise. It will be further understood that the terms “comprise”, “comprises”, “comprising”, “include”, “includes”, “including”, and/or “having.” when used herein, can specify the presence of stated features, integers, steps, operations, elements, and/or components, but do not preclude the presence or addition of one or more other features, integers, steps, operations, elements, components, and/or groups thereof. As used herein, the phrase “in an embodiment” does not necessarily refer to the same embodiment, although it may. As used herein, the phrase “in one embodiment” does not necessarily refer to the same embodiment, although it may. As used herein, the phrase “in another embodiment” does not necessarily refer to a different embodiment, although it may. Further, embodiments and/or components of embodiments can be freely combined with each other unless they are mutually exclusive.

The corresponding structures, materials, acts, and equivalents of all means or step plus function elements, if any, in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A computer-implemented method comprising:
receiving data associated with a patient undergoing a medical treatment, the data pertaining to use of the medical treatment and a state of the patient;
identifying a subset of the data pertaining to the state of the patient as context;
learning a function that relates the context to a reward derived from the medical treatment; and
using the function based on a current state of the patient to identify a type of treatment to deliver to the patient.

2. The method of claim 1, wherein the medical treatment is a self-administered medical treatment where the medical treatment is initiated in clinic and subsequently self-applied by the patient.

3. The computer-implemented method of claim 1, wherein the medical treatment is performed using a wearable medical device that the patient is wearing.

4. The computer-implemented method of claim 1, wherein the reward is related to a metric structured as a multi-dimensional vector that describes the state of the patient related to the medical treatment the patient is undergoing.

5. The computer-implemented method of claim 4, wherein the reward is a scalar function of the context.

6. The computer-implemented method of claim 1, wherein the subset of data includes a set of features from the received data.

7. The computer-implemented method of claim 1, wherein the function is learned using a contextual multi-armed bandit robust with respect to super-gaussian noise.

8. A computer program product comprising a computer readable storage medium having program instructions embodied therewith, the program instructions readable by a device to cause the device to:
receive data associated with a patient undergoing a medical treatment, the data pertaining to use of the medical treatment and a state of the patient;
identify a subset of the data pertaining to the state of the patient as context;
learn a function that relates the context to a reward derived from the medical treatment; and
use the function based on a current state of the patient to identify a type of treatment to deliver to the patient.

10. The computer program product of claim 8, wherein the medical treatment is a self-administered medical treatment where the medical treatment is initiated in clinic and subsequently self-applied by the patient.

11. The computer program product of claim 8, wherein the medical treatment is performed using a wearable medical device that the patient is wearing.

12. The computer program product of claim 8, wherein the reward is related to a metric structured as a multi-dimensional vector that describes the state of the patient related to the medical treatment the patient is undergoing.

13. The computer program product of claim 12, wherein the reward is a scalar function of the context.

14. The computer program product of claim 8, wherein the subset of data includes a set of features from the received data.

15. The computer program product of claim 8, wherein the function is learned using a contextual multi-armed bandit robust with respect to super-gaussian noise.

16. A system comprising:
at least one processor; and
at least one memory device coupled with the at least one processor;
the at least one processor configured to at least:
receive data associated with a patient undergoing a medical treatment, the data pertaining to use of the medical treatment and a state of the patient;
identify a subset of the data pertaining to the state of the patient as context;
learn a function that relates the context to a reward derived from the medical treatment; and
use the function based on a current state of the patient to identify a type of treatment to deliver to the patient.

17. The system of claim 16, wherein the medical treatment is a self-administered medical treatment where the medical treatment is initiated in clinic and subsequently self-applied by the patient.

18. The system of claim 16, wherein the medical treatment is performed using a wearable medical device that the patient is wearing.

19. The system of claim 16, wherein the reward is related to a metric structured as a multi-dimensional vector that describes the state of the patient related to the medical treatment the patient is undergoing.

20. The system of claim 16, wherein the function is learned using a contextual multi-armed bandit robust with respect to super-gaussian noise.
